(19) United States
(12) Patent Application Publication
(10) Pub. No.: US 2018/0140640 A1
(43) Pub. Date: May 24, 2018

(54) IMPROVED CONTRACEPTIVE METHODS AND COMPOSITIONS AND DEVICES FOR USE THEREIN

(71) Applicant: Cadens Beheer B.V., Kootwijk, (NL)

(72) Inventor: Mark Hans EMANUEL, Aerdenhout (NL)

(73) Assignee: Cadens Beheer B.V., Kootwijk, (NL)

(21) Appl. No.: 15/568,031

(22) PCT Filed: Apr. 22, 2015
(86) PCT No.: PCT/NL2015/050268

§ 371 Date: Oct. 20, 2017

(51) Int. Cl. A61K 35/50; A61K 9/00; A61P 15/18; A61F 6/14

(52) U.S. Cl. CPC A61K 35/50; A61F 6/14; A61P 15/18; A61K 9/0039

(57) ABSTRACT

The invention pertains to improved contraceptive methods based on a reversible pseudo state of the Asherman's syndrome, in which the female body follows the normal natural part of the menstrual cycle that occurs in the ovary, the ovulatory cycle, and as a consequence the emotional life follows the normal mental passion patterns, but in which the normal uterine cycle and endometrium functions have been effectively switched off and the uterus resides in a quiescent phase preventing pregnancy and menstruation. The invention can also be used for preventing or suppressing abnormal endometrial bleeding.

### TECHNICAL FIELD

The present invention relates to an improved method of contraception, and for suppression of endometrial bleeding, the method having superior aspects over conventional contraceptive methods such as those based on progestogenic activity, and the special aspect that the natural emotional life is preserved. The invention also relates to intrauterine devices and compositions for use in such methods.

### BACKGROUND OF THE INVENTION

Birth control, also known as contraception and fertility control comprises methods or devices used to prevent pregnancy. Planning, provision and use of birth control is needed for active family planning. Family planning is one of the fundaments of modern society. Birth control increases economic growth due to the fact that there are fewer dependent children and thus more women participating in the workforce. By lengthening the time between pregnancies, birth control can improve adult women health and delivery outcomes as well as the survival of their children. Earnings of women, their body mass index, and their children schooling and body mass index all improve with greater access to birth control. Family planning via the use of modern birth control is one of the most cost-effective health interventions.

A wide range of methods for contraception are known and in current use. Sterilization, while highly effective, is usually not reversible; other methods such as fertility awareness methods, condoms and diaphragms are reversible.

Also commonly used are intrauterine devices (IUD) denoted as IUDs, which work effectively during a long period of time. Examples are described in US2014/261444 and EP2140860 (their contents herein incorporated by reference), the latter discloses an intrauterine delivery system for contraception which prevents or suppresses abnormal and irregular endometrial bleeding. Progestogen-based compounds or compounds having progestogenic activity are used. The compound Danazol® is described, a synthetic steroid which suppresses the estrogen and progesterone receptors in the endometrium, leading to endometrial atrophy and thereby reduced menstrual loss and amenorrhoea in some women. This compound however, has weak androgenic properties and a number of claimed undesired side-effects, such as mood changes like depression and anxiety, increase in bodyweight, and a decreased libido.

NZ596005 discloses an intrauterine delivery system for general utilization, for the use in the treatment of dysfunctional uterine bleeding; menorraghia; dysmenorrhoea; endometriosis and the like. The T shaped device frame has an open structure allowing access to a substantial part of an outer surface of a deposit, where the deposit has a release rate-controlling structure. The contents of this publication is herein incorporated by reference.

In use as intrauterine device are T-shaped devices, which are made of flexible plastic and inserted into a woman's uterus to prevent pregnancy. Two types of IUDs are often used, one contains copper and is effective for about 5-10 years and the other is a hormonal IUD which releases progestin and is effective for about 3-5 years.

The most commonly used methods however are based on hormonal contraceptives like progestogen and often administered as oral pills, or sometimes used in the form of vaginal rings or implants, ‘the pill’ comprising progestogen as main active component perhaps being the most profound method with a history of more than 50 years. These are considered the most reliable, safe and effective contraception methods. EP2305267, US2014/050794, AU2014/227490 describe such pharmaceutical progestogen-based compositions for use as a contraceptive in birth control pills. There are two main types of oral birth control pills, the combined oral contraceptive pills which contain both progestogen and estrogen, and the ‘progestogen only’ pills. Irregular bleeding may occur with the progestin only methods, while some users report no periods.

Although the pill is one of the most successful contraceptives with a long history of utilization, there are some concerns regarding long-term side effects. Combined hormonal contraceptives are associated with an increased risk of venous and arterial blood clots. Psychological effects on sexual desire have been reported, some feel an increase, but many others complain about a decrease in sexual desire. The loss of libido is considered to be a psychological effect due to the fact that the hormone progesterone is related to the luteal phase, which is the infertile period following the fertile follicular phase. Another preconception of women is that their body weight increases due to the use of hormones, while other women consider the use of hormones as not naturally and hence undesirable.

Of interest is that there exists a natural birth control method, i.e. breastfeeding or Lactational Amenorrhea Method (LAM), which does not involve pharmaceutical interference. However, it can be only safely be used during the first 6 months after giving birth, when breastfeeding naturally changes the hormonal pattern of a woman in such a way that she has an anovulatory cycle and cannot become pregnant.

In the art there remains a need for an effective contraceptive method which is more generally applicable and reversible, and with as little interference in women's life as possible.

### SUMMARY OF THE INVENTION

The inventor has found the driving force behind Asherman's syndrome (AS) and also found a way to advantageously translate those insights in improved contraceptive methods, devices and compositions. Asherman's syndrome or Fritsch syndrome is a condition characterized by adhesions and/or fibrosis of the endometrium most often associated with dilation and curettage of the intrauterine cavity subsequent to a prior state of pregnancy. Although a normal ovarian cycle can be observed in patients with AS and hormone studies show normal levels consistent with the reproductive function, the endometrium is however not responsive to the stimulus of the hormonal endocrine ovarian cycle and no menstrual pattern and absence of haematometra is normally observed. As the endometrium is unresponsive, in an apparent quiet state and has been effectively switched off, resembling a quiescent state of the uterus, AS results in infertility. AS may easily be clinically resolved. After opening of the uterine cavity, the endometrial function restores immediately including menstrual periods and fertility.

The inventors' investigation of the uterus of AS patients revealed enclosures formed by the intrauterine adhesions which contain microscopic pregnancy residues. The adhesions prevent that the pregnancy residues are disposed of in a normal way. These enclosed AS residues affect the normal function of the endometrium and keep the endometrium in a quiescent phase. On microscopic observation the AS residues were found to comprise residue cells, on some occasions even years after pregnancy ended. These AS residue cells secrete one or more active compounds which induce the quiescent phase of the endometrium thus avoiding menstrual bleeding but preventing pregnancy. The ‘quiescent phase’ refers to the fact that endometrium is in a rest phase, insignificantly or not influenced by the hormones of the ovarian cycle.

Advantageously, by making use of these therapeutically active AS residue cells and/or the active compounds secreted by these AS residue cells the inventors have provided a safe and reversible contraceptive method that does not interfere with the woman's anovulatory cycle and the emotional aspects thereof, and as such can be appreciated as a natural method, in sharp contrast with those traditional hormonal contraceptives which work by disrupting the normal menstrual cycle pattern, the emotional life and the normal mental passion patterns associated with the menstrual cycle.

The invention thus pertains to a reversible contraceptive induced ‘pseudo-AS’ method which comprises a normal emotional life corresponding to the natural menstrual cycle in the ovary, in combination with an uterus which resides in a quiescent state, resulting in absence of menstrual bleeding and preventing pregnancy.

The invention also pertains to a contraceptive implantable medical device that induces a quiescent phase of the endometrium that mimics the quiescent phase initiated by the Asherman's syndrome in a female human subject in terms of maintaining the ovulatory cycle but preventing menstrual bleeding and pregnancy.

The invention also pertains to female uterine AS residue cell cultures, isolated AS residue cells and AS residue secretion and, one or more compounds such as secreted by AS cells, for use in female contraception.

More detail is provided in the detailed description here below.

### LIST OF PREFERRED EMBODIMENTS ACCORDING TO THE INVENTION

1. A method for the sterilization of a female human subject, said method involving bringing therapeutically active AS residue cells and/or one or more active compound(s) such as one or more active compound(s) secreted by AS residue cells in the uterus of said female human subject;
2. the method according to embodiment 1, wherein said method induces a state which mimics Asherman's syndrome in terms of maintaining the ovulatory cycle but preventing menstrual bleeding and pregnancy;
3. the method according to any of the preceding embodiments, wherein said therapeutically active AS residue cells are trophoblast cells;
4. the method according to any of the preceding embodiments, using an intrauterine device comprising or encapsulating said therapeutically active AS residue cells, and bringing said therapeutically active AS residue cells at a position in the uterus suitable for the cells to release or secrete their active compound(s) into the uterus;
5. the method according to any of the preceding embodiments, using an intrauterine device for controlled release of said one or more active compounds such as one or more active compound(s) secreted by AS residue cells, over a prolonged period of time and at a level required for contraception, wherein the intrauterine device comprises a body construction and at least one reservoir comprising a core, wherein said intrauterine device comprises therapeutically effective amounts of one or more active compounds such as one or more active compound(s) secreted by AS residue cells;
6. the method according to any of the preceding embodiments, for use in preventing or suppressing abnormal endometrial bleeding;
7. a contraceptive implantable intrauterine device [IUD] that induces a quiescent phase of the endometrium that mimics the quiescent phase initiated by the Asherman's syndrome in a female human subject in terms of maintaining the ovulatory cycle but preventing menstrual bleeding and pregnancy;
8. the IUD according to embodiment 7, comprising a body or support construction configured for transcervical implantation in a uterus and at least one reservoir comprising therapeutically active AS residue cells and/or therapeutically effective amounts of one or more active compounds such as one or more active compound(s) secreted by AS residue cells, said AS residue cells or compound(s) capable of inducing a quiescent phase and preventing or suppressing endometrial bleeding;
9. the IUD according to embodiment 7 or 8, for use in preventing or suppressing abnormal endometrial bleeding;
10. the method according to any of embodiments 1-6 or the IUD according to any of embodiments 7-9, wherein said therapeutically active AS residue cells are derived from an earlier pregnancy of said female human subject;
11. a female human AS residue cell culture or AS residue secretion or one or more compounds such as one or more active compound(s) secreted by AS residue cells for use in contraception of a female human subject and/or for use in preventing or suppressing abnormal endometrial bleeding in a female human subject; and
12. use of a female human AS residue cell culture or AS residue secretion or one or more compounds such as one or more active compound(s) secreted by AS residue cells in female contraception and/or in the manufacture of a female contraceptive, preferably an intrauterine device.

### DETAILED DESCRIPTION OF THE INVENTION

### Menstrual Cycle

In healthy fertile female humans the menstrual cycle is the cycle of natural changes that occurs simultaneously in the ovary and uterus, and is an essential part of making reproduction possible. The menstrual cycle can be subdivided into the ovarian cycle for the ovary and the uterine cycle for the uterus. The ovarian cycle can be divided into three phases, based on biological events. The ovarian cycle consists of the follicular phase, the ovulation, and the luteal phase, which are essential for the production of eggs, fertilization, and the preparation of the uterus for pregnancy.

The ovarian cycle fully controls the uterine cycle by the release of hormones.

The follicular phase is the first part of the ovarian cycle. During this phase, an ovarian follicle matures and gets ready to release an egg. The follicular phase controls the proliferative phase of the uterine cavity by the release of the hormone estradiol which after the menstruation stimulates the growth of the endometrium.

The second phase of the ovarian cycle is the ovulation, in which an ovarian follicle releases a mature egg towards the ampulla of the oviduct, after which fertilisation must occur within 24 hours, or the unfertilized egg will disintegrate and dissolve in the fallopian tube or abdominal cavity.

The final phase of the ovarian cycle, the luteal phase is characterized by the release of the hormone progesterone by the remains of the ovarian follicle, also called corpus luteum, which induces the secretory phase of the uterine cycle and prepares the uterus for implantation of an embryo and possible pregnancy. The start of the luteal phase also ends the fertile phase of the cycle, progesterone not only prepares the uterus for possible pregnancy but also adds by forming a cervical mucus plug which acts as a protective barrier by deterring the passage of micro-organisms and sperm into the uterus.

Hormones released during the ovarian cycle cause behavioral changes in females, and mild to severe mood changes can occur. A significant correlation between low progesterone levels and the ability to accurately recognize emotion has been found, and as a consequence women in the follicular stage of the ovarian cycle show higher emotional recognition accuracy than their luteal phase counterparts. Women were found to react more strong to negative stimuli when in the luteal phase over the women in the follicular stage, indicating more reactivity to social stress during the second half of the menstrual cycle.

The natural shift of hormone levels during the different phases of the menstrual cycle can be related to the emotional life of a woman who feels she becomes fertile in the follicular stage and performs better in tasks that contain empathetic traits. In the non-fertile luteal phase her empathy decreases, but her ability to cope with social stressful situations is increased. As said before, hormonal contraceptives interfere with these natural shifts.

### Asherman's Syndrome

Asherman's syndrome (AS) or Fritsch syndrome, is a condition characterized by adhesions and/or fibrosis of the endometrium most often associated with dilation and curettage of the intrauterine cavity subsequent to a prior state of pregnancy. In the original description of the syndrome [1] the amenorrhea and the iatrogenic nature of the pathogenesis are emphasized. Earlier the disorder already has been described in 1894 by Heinrich Fritsch [2].

A common misconception found in literature about AS is that it is characterized by intrauterine adhesions of the endometrium only, and cases have incorrectly been reported as AS, characterized by adhesions only without prior state of pregnancy, and these cases have been excluded from the discussion. Those skilled in the field will appreciate that AS is limited to those conditions with adhesions and/or fibrosis of the endometrium following pregnancy.

The cavity of the uterus is lined by the endometrium. This lining is composed of two layers, the functional layer, adjacent to the uterine cavity, which is shed during the menstruation and an underlying basal layer, adjacent to the myometrium, which is necessary for regenerating the functional layer. Trauma to the basal layer, typically after a dilation and curettage performed after a miscarriage, a delivery, or after surgical termination of a pregnancy, can lead to the development of intrauterine scars resulting in adhesions that decrease the functionality of the cavity to varying degrees.

Although a normal ovarian cycle can be observed in patients with the AS and hormone studies show normal levels consistent with the reproductive function, the endometrium is however not responsive to the stimulus of the hormonal endocrine ovarian cycle and no menstrual pattern and absence of haematometra is normally observed. As the endometrium is unresponsive, in an apparent quiet state and has been effectively switched off, resembling a quiescent state of the uterus, AS results in infertility.

In the context of the invention, the term ‘pseudo AS’ is reserved for contraceptive methods providing the female human subject's uterus with therapeutically effective AS residue cells secreting one or more active compounds or compositions comprising one or more of the active compounds secreted by AS residue cells, thus yielding the AS characteristics of rendering the endometrium unresponsive to normal hormonal endocrine ovarian cycle. Pregnancy and menstrual bleeding are prevented, but the ovulatory cycle is maintained.

In the context of the invention, the term ‘AS residue cells’ and ‘pregnancy residue cells’ are used interchangeably, and refer to isolated and/or cultured therapeutically active cells. In one aspect, the AS residue cells are trophoblast cells. Trophoblast cells form the outer layer of a blastocyst in the uterus, which provide nutrients to the embryo and develop into a large part of the placenta. They are formed during the first stage of pregnancy. In the context of the invention, these trophoblast cells are therapeutically active and exhibit secretion abilities. In one embodiment, the trophoblast cells are isolated from human pregnancy residues, and preferably relate to a cell culture comprising trophoblast cells derived from pregnancy residues. In one embodiment, the AS residue cells are derived (cell cultured) from earlier pregnancy residues of said female subject herself. In one embodiment, said AS residue cell secreted or secretable (i.e. ‘obtainable by secretion’) compound(s) comprise human chorionic gonadotropin (hCG) and/or estriol. The term AS residue cell secreted compound(s)—such as hCG and/or estriol—also encompasses compounds such are obtainable by secretion by AS residue cells.

In one embodiment of the invention described in the specification and claims, the ‘one or more active compound(s) secreted by AS residue cells’ encompasses compositions such as secreted or secretable by therapeutically active AS residue cells (i.e. AS residue secretion) when located in the uterus. The compound(s) could be derived from AS residue cells, but also encompass(es) compound(s) such are obtainable by secretion (secretable′) by AS residue cells. The inventors believe that the AS residue secretion or the one or more active compound(s) therein could be applied through oral, intramuscular, transdermal, intranasal, parenteral, sublingual, topical, intra-vaginal, or rectal administration routes, and could be administered in the form of a tablet, pill, dispersed powder, capsule, granule, emulsion, solution, suspension, syrup, suppository, or patch. However, in a preferred embodiment the AS residue secretion and/or one or more active compound(s) secreted or secretable by AS residue cells' therein are applied locally, i.e. in the uterus.

In a first aspect, the invention pertains to a method for the sterilization of a female human subject, said method involving bringing therapeutically active AS residue cells and/or one or more therapeutically active compounds such as secreted by AS residue cells in the uterus of said female human subject. The method involves amounts of these AS residue cells or AS residue-secreted or AS-secretable compound(s) sufficient to render a state which mimics Asherman's syndrome is induced, i.e. a pseudo AS.

In one embodiment the therapeutically active AS residue cells are brought into the uterus surgically, to achieve that these cells invade the uterus in therapeutically effective amounts. The active compound(s) secreted or generated by AS residue cells such as trophoblast cells induce the quiescent uterus phase strived for.

In a preferred embodiment the above may be achieved non-surgically. The method preferably involves inserting or placing an intra-uterine device [IUD] comprising or encapsulating said therapeutically active AS residue cells and/or therapeutically effective amounts of one or more active compounds such as secreted by AS residue cells, said AS residue cells or said compound(s) capable of inducing a quiescent phase and preventing or suppressing endometrial bleeding, at a suitable position in the uterus.

In one embodiment, the IUD is preferably designed for controlled release of the therapeutically active components obtainable by AS residue cell secretion, such as AS residue cell secretion, preferably the controlled release of one or more trophoblast-secreted compounds. Suitable IUDs are available in the art, and are for instance described in EP2140860, its contents herein incorporated by reference. In a preferred embodiment, the invention thus pertains to a method for contraception of a female human subject and mimicking Asherman's syndrome in terms of maintaining the ovulatory cycle but preventing menstrual bleeding and pregnancy, using an intrauterine device for controlled keeping of AS residue cells, preferably trophoblast cells, and/or controlled release of one or more active compounds such as secreted by AS residue cells and which compounds are released over a prolonged period of time and at a level required for contraception, wherein the intrauterine device comprises a body construction and at least one reservoir comprising the active agent(s).

The release of the one or more active compounds secreted by AS residue cells preferably lasts for from one up to ten years, or from one to five years, or preferably from three to five years.

In a second and related aspect, the invention concerns an implantable intra-uterine device that mimics the pregnancy residue in order to initiate a pseudo AS and to be used in the preferred contraceptive methods above. The contraceptive intra-uterine device [IUD] allows a pseudo AS syndrome or state of AS to be induced, which is reversible and can be used as new contraceptive technique to prevent pregnancy while the normal ovulatory cycle is preserved. Preserving the normal ovulatory cycle will lead to the ability of a living a natural non-disturbed emotional life which is generally accepted as a pleasant condition, and is desired by many women.

The IUD comprises a body or support construction configured for transcervical implantation in an uterus and at least one reservoir comprising the AS residue cells and/or one or more active compounds such as secreted by AS residue cells.

The contraceptive device comprises a support structure such as a subcutaneous rod or an insertable intravaginal ring, and a reservoir for the AS residue cells and/or one or more active compounds such as secreted by AS residue cells. Further in such embodiments, the support structure may comprise a polymeric material. The support structure may comprise a T-like, a V-like, a 7-like, a 8-like, a loop-like, a zigzag-like, or a ring-like configuration.

The reservoir may comprise a core and optionally a membrane encasing the core, the core and membrane, said membrane preferably essentially consisting of a same or different polymer composition, wherein said intrauterine device comprises therapeutically active AS residue cells and/or therapeutically effective amounts of one or more active compounds such as secreted by AS residue cells, said AS residue cells or compound(s) capable of inducing a quiescent phase and preventing or suppressing endometrial bleeding. The core is a polymer matrix wherein the therapeutically active substance or substances are dispersed.

The polymer compositions are chosen according to the release rates desired. The release rates can be controlled by the membrane or by the membrane together with the core, but the release rate can also be controlled by the core alone. In principle any polymer, either biodegradable or non-biodegradable, can be used as long as it is biocompatible. As known in the art, the release kinetics of a therapeutically active agent from a polymer based delivery system depends on the molecular weight, solubility, diffusivity and charge of the therapeutically active agent as well as on the characteristics of the polymer, on the percentage of the loading of the therapeutically active agent, on the distance the therapeutically active agent must diffuse through the device body to reach its surface and on the characteristics of any matrix or membrane. Information regarding controlled release IUDs are regarded to fall within the ambit of the skilled person's common general knowledge.

In the aforementioned IUDs, the amount of the therapeutically active AS residue cells and/or the one or more AS residue-secreted substances incorporated in the delivery system, both the AS residue cells and/or the one or more AS residue-secreted substances capable of realizing the pseudo AS state, varies depending on the particular therapeutically active agent and the time for which the intrauterine system is expected to provide therapy. There is no critical upper limit on the amount of therapeutically active agent incorporated in the device since, depending on the selected body construction, the size, shape and number of reservoirs for administering dosages can be varied and modified. The lower limit depends on the efficacy of the therapeutically active agent and the expected release time. It is however observed in the accompanying studies that residue levels of AS residue cells would be sufficient for rendering the desired pseudo AS effects.

In a preferred embodiment, the one or more active compound(s) secreted or secretable or, such as secreted by the AS residue cells is referred to as AS residue secretion.

In a third and related aspect, the invention pertains to a female human AS residue cell culture, isolated AS residue cells or AS residue secretion or, one or more compounds such as secreted by AS residue cells, for use in contraception of a female human subject and/or for use in preventing or suppressing abnormal endometrial bleeding in a female human subject. The invention also pertains to the use of a female human AS residue cell culture, isolated AS residue cells or AS residue secretion in female contraception, and/or in the manufacture of a female contraceptive or a IUD such as mentioned elsewhere in the description.

Other embodiments of this invention will be apparent to those skilled in the art upon consideration of this specification or from practice of the invention disclosed herein. Variations on the embodiments described herein will become apparent to those of skill in the relevant arts upon reading this description. The inventors expect those of skill to use such variations as appropriate, and intend to the invention to be practiced otherwise than specifically described herein. Accordingly, the invention includes all modifications and equivalents of the subject matter recited in the claims as permitted by applicable law. Moreover, any combination of the above described elements in all possible variations thereof is encompassed by the invention unless otherwise indicated.

### Pilot Study

A study was conducted, in which from patients with a normal ovarian cycle and amenorrhoea resulting from the AS, 19 endometrial biopsies were taken from the endometrium of the fundus of the uterine cavity. At the same time blood samples were taken for hormone analysis of the plasma (progesterone, estrogen, FSH, LH). Subsequently, by means of microscopic examination of the sections (hematoxylin and eosin staining), an attempt was made to determine a cycle dating of the endometrium, based on the histological picture of the stroma and glandular tubes by use of the local protocol of the Department of Pathology of the VU University Medical Center. For the detection or exclusion of any disparity or dyssynchrony, the development of the stroma and the maturation of the gland tubes were individually described and dated. This dating was subsequently compared to the plasma levels of the hormones, where the height of the progesterone level gives the best indication with respect to the determination of the moment in the ovarian cycle.

The hormonal receptors were immuno-histochemically stained in the biopsies and were observed to be present in normal amounts.

The results are displayed in the table below. The results are ranked, first the date on the basis of the stroma, then on the basis of the gland ducts, and then on the basis of the height of the progesterone. For 4 biopsies the maturation of the gland tubes could not be determined because too few representative gland ducts were present in the material.

| | cycle | cycle | | | | |
|---|---|---|---|---|---|---|
| | day | day | Progesterone | Estradiol | FSH | LH |
| Biopsy | stroma | glands | nmol/l | pmol/l | U/l | U/l |
| I | 22 | 22 | 3.2 | 272 | 8 | 7 |
| F | 22 | 22 | 10.2 | 244 | 7 | 5 |
| M | 22 | 22 | 35.5 | 939 | 1 | 1 |
| G | 22 | 22 | 38.6 | 318 | 2 | 4 |
| S | 22 | 23 | 17.7 | 420 | 3 | 2 |
| Q | 23 | 24 | 11.1 | 371 | 4 | 3 |
| O | 24 | 23 | 0.8 | 547 | 2 | 1 |
| N | 24 | 24 | 6.1 | 288 | 3 | 3 |
| R | 24 | 24 | 9.7 | 300 | 2 | 1 |
| A | 24 | 24 | 17.3 | 356 | 3 | 2 |
| D | 24 | 24 | 28.3 | 672 | 2 | 2 |
| C | 25 | 17 | 20.8 | 253 | 9 | 4 |
| L | 25 | 20 | 13.0 | 402 | 3 | 4 |
| E | 26 | 16 | 0.6 | 37 | 3 | 2 |
| K | 26 | 20 | 0.9 | 69 | 5 | 3 |
| H | 24 | * | 0.6 | 177 | 8 | 6 |
| J | 24 | * | 0.7 | 256 | 3 | 3 |
| P | 24 | * | 1.2 | 169 | 4 | 3 |
| B | 24 | * | 20.1 | 734 | 4 | 5 |

* could not be determined

All subjects had an amenorrhoea (absence of menstrual bleeding), but normal ovarian cycle, illustrated by the random distribution of the progesterone values, as was expected, and was found. A progesterone value of less than 2.5 to 3.5 nmol/1 is generally considered to be appropriate to proliferation or during menstruation. In none of the biopsies corresponding to such progesterone value, however, a histological picture was seen appropriate for proliferation or menstruation. All biopsies were observed to be in a state of more or less mature secretion.

The endometrium appears to be in a resting phase that is most similar to the second half of the cycle, the postovulatory or secretional phase or luteal phase or decidual phase in which normally the endometrium happens to be at time of the implantation or early pregnancy.

The endometrium in the subjects showed enclosures, formed by the intrauterine adhesions which contain microscopic pregnancy residues. These enclosed residues affect the normal function of the endometrium and keep the endometrium in a quiescent phase. Inspection of the residues revealed cells which secrete endocrine factors.

### NON-PATENT LITERATURE CITED IN THE DESCRIPTION

[1] Asherman J G. Amenorrhea traumatica (atretica). J Obstet Gynaecol Br Emp 1948; 55:23-30.
[2] Fritsch H. Ein Fall von volligen Schwund der Gebaumutterhohle nach Auskratzung. Zentralbl Gynaekol 1894; 18:1337-42.

1.-12. (canceled)

13. A method for the sterilization of a female human subject, comprising administering therapeutically active Asherman's syndrome (AS) residue cells and/or one or more active compound(s) secreted by AS residue cells to the uterus of the female human subject.

14. The method according to claim 13, wherein the method maintains the ovulatory cycle but prevents menstrual bleeding and pregnancy.

15. The method according to claim 13, wherein the therapeutically active AS residue cells are trophoblast cells.

16. The method according to claim 13, wherein the therapeutically active AS residue cells are encapsulated and/or provided in an intrauterine device.

17. The method according to claim 16, comprising administering the therapeutically active AS residue cells at a position in the uterus suitable for the cells to release or secrete their active compound(s) into the uterus.

18. The method according to claim 16, wherein the intrauterine device controls release of the one or more active compounds over a prolonged period of time and at a level required for contraception, wherein the intrauterine device comprises a body construction and at least one reservoir comprising a core harboring the therapeutically active Asherman's syndrome (AS) residue cells and/or one or more active compound(s) secreted by AS residue cells.

19. The method according to claim 13, wherein the administration prevents or suppresses abnormal endometrial bleeding.

20. The method according to claim 13, wherein the therapeutically active AS residue cells are derived from an earlier pregnancy of the female human subject.

21. A contraceptive implantable intrauterine device (IUD), comprising a body or support construction configured for transcervical implantation in a uterus and at least one reservoir comprising therapeutically active AS residue cells and/or therapeutically effective amounts of one or more active compounds such as one or more active compound(s) secreted by AS residue cells, the AS residue cells or compound(s) capable of inducing a quiescent phase and preventing or suppressing endometrial bleeding.

22. The IUD according to claim 21, for use in preventing or suppressing abnormal endometrial bleeding.

23. The method according to claim 21, wherein the therapeutically active AS residue cells are derived from an earlier pregnancy of the female human subject.

24. A female human Asherman's syndrome (AS) residue cell culture or AS residue secretion or one or more compounds such as one or more active compound(s) secreted by AS residue cells for use in contraception of a female human subject and/or for use in preventing or suppressing abnormal endometrial bleeding in a female human subject.

25. A method for preventing or suppressing abnormal endometrial bleeding in a female human subject, comprising administering to the female human subject a therapeutically active Asherman's syndrome (AS) residue cell culture and/or one or more active compound(s) secreted by AS residue cells.
